(19) United States
(12) Patent Application Publication
(10) Pub. No.: US 2005/0276871 A1
(43) Pub. Date: Dec. 15, 2005

(54) Control of canine voluntary food intake

(76) Inventors: Donald M. Mattsson JR., Des Moines, IA (US); Lynn Deffenbaugh, Topeka, KS (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/151,082

(22) Filed: Jun. 13, 2005

Related U.S. Application Data
(60) Provisional application No. 60/579,439, filed on Jun. 14, 2004.

(51) Int. Cl. A61K 35/78

(52) U.S. Cl. 424/757; 424/773

(57) ABSTRACT

Proteinase inhibitors are found to be a satiety aid when orally administered to dogs prior to a meal. Specifically, potato proteinase inhibitor 2 (PI2) by daily oral administration to groups of dogs showed a reduction in daily food intake in a crossover study. Analysis of food intake data during three separate treatment periods showed that animals that received the highest dose of PI2 ate significantly less on average than untreated animals. Analysis of food intake data confirmed that food intake decreased in a dose response manner with PI2 treatment. Therefore, a dietary aid for pets that incorporates PI2 could provide a pet owner with an additional resource to help their pet consume less food.

### BACKGROUND OF THE INVENTION

1. Field of the Invention

The invention relates generally to the control of voluntary food intake in dogs and, more specifically, to the oral administration of a proteinase inhibitor to dogs prior to a meal which results in the voluntary reduction of food intake by the dog. By voluntarily decreasing food intake, dogs administer the proteinase inhibitor are likely to lose weight.

2. Background of the Art

Obesity in pets is a significant veterinary health issue that manifests in over 40% of dogs and cats that are seen by veterinarians. MarketSense, Inc. July 2003. Small animal veterinarian attitudes and behaviors regarding nutritional products: a qualitative marketing research study. Most obesity is due to over feeding at mealtime and to owners providing unnecessary snacks to their pets. Unfortunately, veterinarians estimate that they have been successful in treating obesity in only about 10% of cases. One reason for the lack of success might be the difficulty that pet owners have with compliance to a strict feeding program. Pet owners that wish to help their pet lose weight might find a satiety aid very helpful in achieving their goal. The satiety aid, when ingested by the animal before feeding, would result in the animal feeling satisfied sooner during a meal and therefore consuming less pet food.

Nutrition to counteract obesity can be divided into two main areas. First, ways to prevent animals from becoming overweight and second, management of a weight reduction program. Regulation of energy intake is a complex physiological system that includes food intake, nutrient turnover, thermogenesis and body fat mass. In addition, there may be a genetic predisposition for obesity. Animal nutritionists generally recognize the need to reduce energy supply and increase energy utilization as the key components to maintain or reduce body fat. However, there is also a need to alter animal behavior, metabolic systems and feedback mechanisms. Consequently, once a pet owner has become convinced their animal is overweight it is difficult for them to conform to an effective dietary program over a long period in order to achieve steady weight loss.

Cholecystokinin (CCK) is a gastrointestinal peptide hormone that is released from the digestive tract into the circulatory system after consuming food and acts to control short-term food intake. Smith G P and J Gibbs. 1975. Cholecystokinin and satiety: theoretic and therapeutic implications. In Hunger: Basic Mechanisms and Clinical Implications. D Novin, et al, eds. Raven Press, New York. Release of CCK from the digestive tract is stimulated by protein and inhibited by endogenous proteases such as trypsin. Malfertheiner P, B Glasbrenner and M Buchler. 1988. Role of nutrients in pancreatic adaptation with implication of Cholecystokinin release. Scandinavian Journal of Gastroenterology Supplement 151:108-113; Miyasaka K, D F Guan, R A Liddle and G M Green. 1989. Feedback regulation by trypsin; evidence for intraluminal CCK-releasing peptide. American Journal of Physiology 257:G175-G181. Normally, following a meal, the human circulating CCK level can reach 8 pmol/L then decline to a pre-meal level after about 40 minutes. Guan D, W T Phillips, J G Schwartz, E Paul, J Rehfeld and G M Green. 1995. Plasma CCK and secretin levels are elevated in type II diabetes. Gastroenterology 108 (4^{th} Supplement):A973. In the absence of proteases, an endogenous releasing factor interacts with cells that produce CCK resulting in stimulation of CCK release. Lu L, D Louie and C Owyang. 1989. A Cholecystokinin releasing peptide mediates feedback regulation of pancreatic secretion. American Journal of Physiology 256:G430-G435.

Potato proteinase inhibitor 2 (PI2) is a potent protease inhibitor that is derived from Russet-Burbank potatoes. Melville J C and C A Ryan. 1972. Chymotrypsin inhibitor I from potatoes. Large scale preparation and characterization of its subunit components. Journal of Biological Chemistry 247:3445-3453. When taken orally by humans, PI2 decreases food intake, induces satiety and can result in weight loss. Hill A J, S R Peikin, C A Ryan and J E Blundell. 1990. Oral administration of proteinase inhibitor II from potatoes reduces energy intake in man. Physiology and Behavior 48:241-246; Schwartz J G, D Guan, G M Green and W T Phillips. 1994. Treatment with an oral proteinase inhibitor slows gastric emptying and acutely reduces glucose and insulin levels after a liquid meal in type II diabetic patients. Diabetes Care 17:255-262; Spiegel T A C Hubert and S R Peikin. 1999. Effect of a premeal beverage containing a protease inhibitor from potatoes on satiety in dieting overweight women. Presented at the Annual Meeting of the North American Association for the Study of Obesity (NAASO); Vasselli J R, D Greenfield, L Schwartz and S B Heymsfiled. 1999. Consumption of a pre-meal drink containing protease inhibitor from potatoes decreases hunger and increases fullness in overweight subjects following a meal. Presented at the Annual Meeting of the North American Association for the Study of Obesity (NAASO). Ingestion of PI2 prior to a meal results in near doubling of the circulating CCK level and feelings of satiety appear to be maintained for a longer period of time. Schwartz, et al.; Spiegel, et al.; Vasselli, et al.

Canine digestive physiology is very similar to humans, particularly with respect to CCK release and serum levels after food consumption. The basal CCK level is about 2 pmol/L which rises to peaks of 5-11 pmol/L within 30-45 minutes of the start of feeding. Eysselein V E, G A Eberlein, W H Hesse, M V Singer, H Goebell and J R Reeve, Jr. 1987. Cholecystokinin-58 is the major circulating form of Cholecystokinin in canine blood. The Journal of Biological Chemistry 262:214-217; Lindén A and K Uvnäs-Moberg. 1987. Plasma levels of Cholecystokinin (CCK-8 and CCK-33-39) in response to feeding and during pregnancy in dogs. Scandinavian Journal of Gastroenterology 22:859-864. The serum CCK level then declines to the basal level about 120 minutes after the start of feeding. While exogenous CCK delays gastric emptying, it is not clear whether the increased serum level of CCK following feeding is sufficient to induce satiety. Keinke O, H J Ehrlein and S Wulschke. 1986. Mechanical factors regulating gastric emptying examined by the effects of exogenous cholecystokinin and secretin on canine gastroduodenal motility. Canadian Journal of Physiology and Pharmacology 65:287-292; Reidelberger R D, T J Kalogeris and T E Solomon. 1989. Plasma CCK levels after food intake and infusion of CCK analogues that inhibit feeding in dogs. American Journal of Physiology 256:R1148-R1154. U.S. Pat. No. 4,833,128 describes a dietary supplement for administration to a mammal prior to a meal as a preload unit to induce satiety and inhibit feeding. The preload is balanced so that it inhibits gastric emptying to a defined rate after a meal. Also claimed is a method to treat obesity in mammals by administering the supplement. The formula of the supplement contains L-phenylalanine, protein, carbohydrate, fat and fiber. Although not claimed, the invention is described as being able to induce the release of CCK which results in a satiety response.

Methods that have been used for calorie reduction in pets, outside of starvation, have included novel dietary ingredients that alter digestion or metabolism, or that result in modified energy partitioning post digestion. Those ingredients include high fiber diets, low calorie fats, inert dietary components, digestion inhibitors, L-carnitine, vitamin A, chromium, biotin, conjugated linoleic acid (CLA), glutamine and several homeopathic compounds.

### SUMMARY OF THE INVENTION

The present invention comprises the use of dietary proteinase inhibitor, such as potato proteinase inhibitor 2 or Bowman-Birk inhibitor from soybeans, in dogs as a treatment to modify daily food intake.

In studies described in this application, six groups of animals were treated separately each day prior to feeding and daily food intake by each animal was measured. Three different treatments included a no-treatment control and two different dosage levels of a preparation that contained PI2. Three successive, separate treatment periods were used and the order of treatments was varied between the groups of dogs so that each group received all possible combinations of the three treatments during the three periods. At the end of the study, food intake data were analyzed for differences between treatments. The study demonstrated that dietary proteinase inhibitors decreased canine food intake in a dose-dependent manner.

### BRIEF DESCRIPTION OF THE DRAWINGS

FIG. 1 is a graphical representation of a comparison of average food intake by animals that received no dietary PI2 during each of the three treatment periods wherein the animals were treated and fed separately and feeding was limited to 45 min each day for five days during each period and different animals received Treatment A during each treatment period.

### DETAILED DESCRIPTION OF A PREFERRED EMBODIMENT

Experiment 1

Materials and Methods

A. Test Material

A potato extract containing 9.44% potato proteinase inhibitor II (PI2) was used as the source of proteinase inhibitor for this study. The potato extract is prepared following the teachings of U.S. Pat. No. 6,767,566 and U.S. application 2003/0077265, which are incorporated herein by this reference. The actual preparation administered to the animals is hereinafter referred to as “PI2 Prep” and contained 43.69% potato extract, 55.76% microcrystalline cellulose, 0.05% silicon dioxide, and 0.5% magnesium stearate. While potato proteinase inhibitor is the only proteinase inhibitor tested in these studies, the term proteinase inhibitor as used herein includes other proteinase inhibitors, specifically Bowman-Birk inhibitor typically extracted from soybeans.

B. Study Design

The pilot study was conducted at an independent research facility. Twenty-four purpose-bred Beagle dogs were used for the study. The dogs were randomly divided into six groups of four dogs. Each group of dogs received treatment for five successive days during each of three treatment periods, with a one-day adaptation between each period during which no treatments were given. Animals were treated orally each day during the test periods and treatments consisted of: A, no PI2 Prep; B, 4 mg PI2 Prep/kg-body weight (bw); and C, 20 mg PI2 Prep/kg-bw. The dogs were weighed before the start of the study and capsules that contained PI2 Prep were prepared for each dog based in the starting weight. Each animal was identified by a unique ear tattoo and matching cage card.

Treatments were administered manually 30 minutes prior to offering food each day and were followed with a 5 ml flush of water. The diet chosen for the study was Iams® Chunks, which was offered to the dogs individually for 45 minutes each day and was the sole source of food for all animals for the length of the study. Although the animals were housed in pairs, they were treated and fed separately.

Food consumption was measured each day by weighing individual food bowls before and after feeding. The order of treatments was varied between the groups of dogs so that each group received all possible combinations of the three treatments during the three treatment periods (Table 1). The study protocol was reviewed and approved by the Institutional Animal Care and Use Committee of the independent research facility prior to initiation of the study and was in compliance with the Animal Welfare Act. Body weights of individual dogs were measured on several days through the course of the study and compared to the clinical end point of 10% weight loss. In addition, qualified personnel performed clinical observations twice a day through the course of the study. No animals were removed from the study due to adverse clinical observations.

TABLE 1
Assignment of dogs to treatment groups and order
of treatments during the three treatment periods.

| | Dog | | | |
|---|---|---|---|---|
| | ID | Treatment^{c} | | |
| Group^{a} | Number^{b} | Days 2-6^{d} | Days 8-12 | Days 14-18 |
| 1 | 10859 | A | B | C |
| | 6956 | A | B | C |
| | 10113 | A | B | C |
| | 10266 | A | B | C |
| 2 | 10639 | A | C | B |
| | 10056 | A | C | B |
| | 8679 | A | C | B |
| | 9098 | A | C | B |
| 3 | 10022 | B | A | C |
| | 10079 | B | A | C |
| | 9802 | B | A | C |
| | 9662 | B | A | C |
| 4 | 3865 | B | C | A |
| | 8345 | B | C | A |
| | 7508 | B | C | A |
| | 9168 | B | C | A |
| 5 | 11011 | C | A | B |
| | 8307 | C | A | B |
| | 7978 | C | A | B |
| | 6046 | C | A | B |
| 6 | 9154 | C | B | A |
| | 8881 | C | B | A |
| | 10077 | C | B | A |
| | 5792 | C | B | A |

^{a}Twenty-four animals were randomly assigned to six different groups.
^{b}Each animal was identified by a unique ear tattoo and matching cage card.
^{c}Treatments consisted of: A, no PI2 Prep; B, 4 mg PI2 Prep/kg-bw; and C, 20 mg PI2 Prep/kg-bw.
^{d}Treatments were administered for five consecutive days during each of three treatment periods with a one-day adaptation between each period during which no treatments were given.

C. Data Analysis

The mean and standard deviation were calculated for total food consumption by all animals for each of the three treatments. The mean and standard deviation were also calculated for food consumed by groups of animals that received each treatment during each treatment period. Equality of the means was tested by ANOVA analysis using General Linear Models software. Differences between pairs of means were tested using a t-test to generate a P value.

Results and Discussion

General trends in the data and statistical analysis of the data showed that food intake by the dogs decreased with increasing dietary PI2. The raw data are included in Appendix I of this application. Data for individual dogs showed a mixed response with some animals eating less with increasing dosage and some animals eating more with increasing dosage (Table 2). The trend from Treatment A to Treatment B to Treatment C showed that five dogs of the twenty-four had a steady decrease in average consumption while two dogs had a steady increase in consumption. Six dogs increased average consumption between Treatment A and Treatment B, then decreased their average food intake during Treatment C to an amount that was below the average for Treatment A. Five other dogs also increased average consumption between Treatment A and Treatment B, then decreased their average food intake during Treatment C to an amount that was above the average for Treatment A. Finally, six dogs decreased average food consumption from Treatment A to Treatment B, then increased average food consumption during Treatment C to an amount that was below the Treatment A average.

TABLE 2
Average daily food consumption by individual
dogs for three different treatments.

| | | Food consumption | | |
|---|---|---|---|---|
| | | (g/day)^{b} | | |
| Group | Dog ID^{a} | A^{c} | B | C |
| 1 | 10859 | 400^{d} | 344 | 302 |
| | 6956 | 375 | 266 | 328 |
| | 10113 | 370 | 225 | 260 |
| | 10266 | 236 | 246 | 240 |
| 2 | 10639 | 298 | 206 | 212 |
| | 10056 | 294 | 275 | 278 |
| | 8679 | 215 | 191 | 154 |
| | 9098 | 255 | 268 | 252 |
| 3 | 10022 | 225 | 400 | 109 |
| | 10079 | 280 | 378 | 223 |
| | 9802 | 199 | 296 | 185 |
| | 9662 | 212 | 177 | 165 |
| 4 | 3865 | 140 | 296 | 189 |
| | 8345 | 100 | 176 | 155 |
| | 7508 | 192 | 372 | 261 |
| | 9168 | 239 | 214 | 223 |
| 5 | 11011 | 329 | 318 | 247 |
| | 8307 | 228 | 261 | 117 |
| | 7978 | 246 | 240 | 314 |
| | 6046 | 283 | 286 | 137 |
| 6 | 9154 | 146 | 163 | 251 |
| | 8881 | 106 | 220 | 233 |
| | 10077 | 318 | 240 | 233 |
| | 5792 | 287 | 209 | 229 |

^{a}Animals were treated and fed separately.
^{b}Food bowls were weighed before and after the 45-min feeding period.
^{c}Treatment A was no PI2 Prep, treatment B was 4 mg PI2 Prep/kg-bw and treatment C was 20 mg PI2 Prep/kg-bw.
^{d}Data were collected and averaged over five days for each treatment period.

The average amount of food eaten by each group of dogs during each treatment period and for each treatment is shown in Table 3. Using the total for each treatment from all three treatment periods, the average daily food consumption decreased in response to increasing dosage of PI2. Average daily consumption by dogs that received Treatment A (no treatment) was 249±99 g. Dogs that received treatment B, 4 mg PI2 Prep/kg-bw, consumed on average 261±91 g food/day, which was no different from Treatment A (P=0.24). In contrast, dogs given Treatment C, 20 mg PI2 Prep/kg-bw, ate 221±88 g food on average per day, which was significantly different from dogs that received no treatment (P=0.006).

TABLE 3
Average amount of food consumed for each
treatment during each treatment period.

| | Treatment Period^{b} | | | |
|---|---|---|---|---|
| Treatment^{a} | 1 | 2 | 3 | Total |
| A | 305 ± 97^{c} | 250 ± 70 | 191 ± 93^{d} | 249 ± 99 |
| B | 289 ± 113 | 239 ± 81 | 256 ± 69 | 261 ± 91 |
| C | 220 ± 117 | 215 ± 64 | 227 ± 74 | 221 ± 88^{e} |

^{a}Treatment A was no PI2 Prep, treatment B was 4 mg PI2 Prep/kg-bw and treatment C was 20 mg PI2 Prep/kg-bw.
^{b}Three successive five-day treatment periods were separated by one-day washout period. Different groups of animals were given different treatments during the three periods as described in Materials and Methods.
^{c}Food intake data as g/day were averaged for each group of animals receiving each treatment in each five-day period.
^{d}Significantly different when compared to Treatment A, period 1; P = 0.001.
^{e}Significantly different when compared to Treatment A Total; P = 0.006.

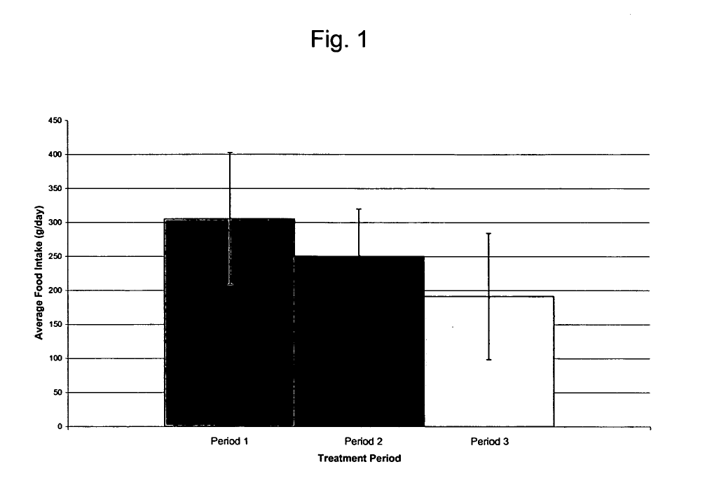

The average amount of food eaten daily by animals that received Treatment A decreased steadily over the course of the three treatment periods (Table 2 and FIG. 1). Initially, during the first treatment period, the untreated control animals each consumed 305±97 g food daily. During the second treatment period the average daily food consumption by the untreated animals decreased, but not significantly, to 250±70 g food (P=0.34). However, during the third treatment period the average daily food intake by untreated animals decreased further to 191±93 g, which was significantly different from daily food intake by untreated animals during treatment period one (P=0.001).

The experiments reported here incorporated a crossover design such that all test animals received all three treatments over the course of three separate treatment periods. Some groups of animals received no treatment during the first treatment period followed by increasing doses of PI2 during the next two treatment periods. Other groups of animals received the highest dose of PI2 during the first treatment period followed by the lower dose and no treatment during the subsequent periods. Therefore, groups of animals that were not treated during the second or third treatment periods had received PI2 treatment, at either a high or low dose, during the preceding treatment periods and might have still been subject to the effects of PI2.

A one-day washout between each treatment period, on which no animals received any treatment, had been included in the experimental design to eliminate the effect of previous PI2 treatments on daily food intake during subsequent treatment periods. If the washout had been effective, the average daily food intake for animals receiving no treatment would not have been different from treatment period to treatment period. The observed daily food intake for animals receiving no treatment did decrease significantly from treatment period to treatment period, which suggested that the washout period was too short and that food intake by the animals was still being affected by previous PI2 treatments.

Groups of animals had not received any treatments prior to treatment period one. Therefore, daily food intake by the animals during that period could not have been affected by previous PI2 treatments and data from treatment period one reflects most reliably the effect of dietary PI2 on daily food intake by dogs (Table 3). During treatment period one, untreated animals on average consumed 305±97 g food daily while PI2-treated animals consumed 289±113 g and 220±117 g food daily when treated with the low and high doses of PI2, respectively. Food intake by animals given the low dosage of PI2 was not different from that of untreated animals or animals given the high dosage of PI2. However, animals that were given the high dosage of PI2 during the first treatment period consumed significantly less food on average than animals that received no treatment (P≦0.05). In addition, the overall decrease in food consumption by animals during the course of the study suggests that PI2 has efficacy over a long term.

In conclusion, the pilot study reported here showed that dietary PI2 decreased canine food intake in a dose-dependent manner. Although the washout period between treatment periods was too short, data from the first treatment period were free from any carry-over effect of PI2 treatment. Also, during treatment period one, PI2 appeared to induce satiety because four animals consumed nothing or only a few grams of food on some of the treatment days. Future studies will need to establish the optimum PI2 dosage level and investigate the long-term affect of dietary PI2 on canine food intake and obesity.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

1. A nutritional intervention composition for reducing voluntary food consumption in companion animals, comprising administration of proteinase inhibitor.

2. The composition of claim 1, wherein the administration is oral.

3. The composition of claim 1, wherein the administration is prior to a meal.

4. The composition of claim 1, wherein the proteinase inhibitor is selected from the group consisting of potato proteinase inhibitor 2 and soybean Bowman-Birk inhibitor.

5. A method of reducing voluntary food intake in a companion animal, comprising the step of administering proteinase inhibitor to the animal.

6. The method of claim 5, wherein the administration is oral.

7. The method of claim 5, wherein the administration is prior to a meal.

8. The method of claim 5, wherein the proteinase inhibitor is selected from the group consisting of potato proteinase inhibitor 2 and soybean Bowman-Birk inhibitor.
